(19) United States
(12) Patent Application Publication
(10) Pub. No.: US 2014/0107458 A1
(43) Pub. Date: Apr. 17, 2014

(54) Resilient Sensor for Biopotential Measurements

(71) Applicants: Maria Op de Beeck, Leuven (BE); Filip Vanlerberghe, O.L.V. Waver (BE)

(72) Inventors: Maria Op de Beeck, Leuven (BE); Filip Vanlerberghe, O.L.V. Waver (BE)

(73) Assignees: KATHOLIEKE UNIVERSITEIT LEUVEN, KU LEUVEN R&D, Leuven (BE); IMEC, Leuven (BE)

(21) Appl. No.: 14/118,543

(22) PCT Filed: May 12, 2012
(86) PCT No.: PCT/EP2012/059237

§ 371 Date: Nov. 18, 2013

Related U.S. Application Data
(60) Provisional application No. 61/488,047, filed on May 19, 2011.

(30) Foreign Application Priority Data
May 18, 2011 (EP) 11166599.8

(51) Int. Cl. A61B 5/0408; A61B 5/0478

(52) U.S. Cl. CPC A61B 5/04087; A61B 5/04085; A61B 5/0478
USPC 600/391; 600/393; 600/372

(57) ABSTRACT

A sensor for biopotential measurement, comprising: an electrical contacting unit for establishing an electrical contact with an animal or human skin, the electrical contacting unit being resilient. The sensor comprises a housing comprising a cavity wherein the electrical contacting unit is partially secured, and means for maintaining said electrical contacting unit in a resiliently deformed state when in contact with said skin.

The present disclosure relates to a sensor for accurate biopotential measurements and to devices comprising the same.

### BACKGROUND TO THE DISCLOSURE

Monitoring of vital health parameters such as electrocardiographic (ECG) or electroencephalographic (EEG) parameters is an important topic in the medical engineering field. Ambulatory measurements of such signals are gaining more and more interest. However ambulatory patients are more mobile than patients in the hospital, resulting in electrode-skin contact related problems. Motion artifact is defined as the noise introduced in the biopotential signal resulting from the relative motion of the measurement electrode and the skin. The electrode movement causes deformation of the skin around it creating changes in the electrical characteristics of the skin. These electrical changes are then registered in the recorded signal as a motion artifact. As motion artifacts are difficult to remove from the signal by for instance a signal processing algorithm, solutions focusing on the sensor design have been proposed.

US2003050550 discloses a dry physiological recording electrode comprising a substrate having an upper and a lower surface, and at least one penetrator(s) protruding from the upper surface of the substrate and wherein the penetrator(s) is capable of piercing through the stratum corneum. The penetrators are said to “lock” the electrode into the chosen skin region and thus reduce motion artifacts.

US2005154273 discloses a body surface biopotential sensor, and an apparatus for detecting biomedical signals. The body surface biopotential sensor of US '273 includes a flexible membrane having a wire layer, and a plurality of electrodes attached on a first surface of the membrane at predetermined intervals. A plurality of needles may be provided on the electrodes to improve an electrical contact stability.

A need still exists for sensors for biopotential measurements which further reduce possible motion artifacts.

### SUMMARY OF THE DISCLOSURE

It is an object of the present disclosure to provide a sensor for accurate biopotential measurements.

This object is met with the means according to the independent claims of the present disclosure. The dependent claims relate to preferred embodiments.

In a first aspect, the present disclosure relates to a sensor for biopotential measurement comprising:
an electrical contacting unit for establishing an electrical contact with an animal or human skin, the electrical contacting unit being resilient so that if pressed against the skin, the electrical contacting unit can resiliently deform over a first distance D in the direction of the pressure P, the electrical contacting unit comprising at least one electrode comprising a first surface for contacting the skin, and
a housing comprising a second surface and a cavity within the second surface, wherein the electrical contacting unit is within the cavity in such a way that the first surface of the electrode is outside of the cavity at a second distance D′, smaller or equal than the first distance D, from the geometrical plane comprising the second surface of the housing, and
means for maintaining said electrical contacting unit in a resiliently deformed state when in contact with said skin.

In a second aspect, the present disclosure relates to a device comprising one or more sensors according to the first aspect.

In a third aspect of the present disclosure, one or more electrical contacting units as described in any embodiment of the first aspect of the present disclosure can be secured in a headset.

In a fourth aspect, the present disclosure relates to the use of a sensor according to the first aspect, of a device according to the second aspect or to a headset according to the third aspect for measuring a biopotential such as a EEG or a ECG.

Particular and preferred aspects of the disclosure are set out in the accompanying independent and dependent claims. Features from the dependent claims may be combined with features of the independent claims and with features of other dependent claims as appropriate and not merely as explicitly set out in the claims.

Although there has been constant improvement, change and evolution of devices in this field, the present concepts are believed to represent substantial new and novel improvements, including departures from prior practices, resulting in the provision of more efficient, stable and reliable devices of this nature.

The above and other characteristics, features and advantages of the present disclosure will become apparent from the following detailed description, taken in conjunction with the accompanying drawings, which illustrate, by way of example, the principles of the disclosure. This description is given for the sake of example only, without limiting the scope of the disclosure. The reference figures quoted below refer to the attached drawings.

### BRIEF DESCRIPTION OF THE DRAWINGS

Further features of the present disclosure will become apparent from the drawings, wherein:

FIG. 1a and FIG. 1b are a schematic representation of the cross-section of a sensor according to an embodiment of the disclosure.

FIG. 2a and FIG. 2b are a schematic representation of different configurations of a sensor according to an embodiment of the disclosure.

FIG. 3 is an enlarged portion of FIG. 1a wherein distance D′ is defined.

FIG. 4 shows the resilient deformation of a electrical contacting unit according to an embodiment of the present disclosure. It defines distance D.

### DETAILED DESCRIPTION OF EMBODIMENTS

The present disclosure will be described with respect to particular embodiments and with reference to certain drawings but the disclosure is not limited thereto but only by the claims. The drawings described are only schematic and are non-limiting. In the drawings, the size of some of the elements may be exaggerated and not drawn on scale for illustrative purposes.

Where an indefinite or definite article is used when referring to a singular noun e.g. “a” or “an”, “the”, this includes a plural of that noun unless something else is specifically stated.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present disclosure, the only relevant components of the device are A and B.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the disclosure described herein are capable of operation in other orientations than described or illustrated herein.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

In the drawings, like reference numerals indicate like features; and, a reference numeral appearing in more than one figure refers to the same element.

In a first aspect, the present disclosure relates to a sensor for biopotential measurement comprising:
an electrical contacting unit for establishing an electrical contact with an animal or human skin, the electrical contacting unit being resilient so that if pressed against the skin, the electrical contacting unit can resiliently deform over a first distance in the direction of the pressure, the electrical contacting unit comprising at least one electrode comprising a first surface for contacting the skin, and
a housing comprising a second surface and a cavity within the second surface, wherein the electrical contacting unit is within the cavity in such a way that the first surface of the electrode is outside of the cavity at a second distance, smaller or equal than the first distance, from the geometrical plane comprising the second surface of the housing, and
means for maintaining said electrical contacting unit in a resiliently deformed state when in contact with said skin.

In other words, in the first aspect, the present disclosure relates to a sensor for biopotential measurement comprising:
an electrical contacting unit for establishing an electrical contact with an animal or human skin, the electrical contacting unit being resilient so that if pressed against the skin, the electrical contacting unit can resiliently deform over a first distance in the direction of the pressure, the electrical contacting unit comprising at least one electrode comprising a first surface for contacting the skin, and
a housing comprising a second surface and a cavity within the second surface, wherein the electrical contacting unit is partially within the cavity in such a way that the first surface of the electrode is outside of the cavity at a second distance, smaller or equal than the first distance, from the geometrical plane comprising the second surface of the housing, and
means for maintaining said electrical contacting unit in a resiliently deformed state when in contact with said skin.

An advantage of a sensor in accordance with embodiments of the present disclosure is that it offers a solution for the problem of motion artifact.

In an embodiment, said means may comprise a skin adhesive present on at least a portion of said second surface of said housing.

In an embodiment, the first aspect of the present disclosure may therefore relate to a sensor for biopotential measurement comprising:
an electrical contacting unit for establishing an electrical contact with an animal or human skin, the electrical contacting unit being resilient so that if pressed against the skin, the electrical contacting unit can resiliently deform over a first distance in the direction of the pressure, the electrical contacting unit comprising at least one electrode comprising a first surface for contacting the skin, and
a housing comprising a second surface and a cavity within the second surface,

wherein the electrical contacting unit is within the cavity in such a way that the first surface of the electrode is outside of the cavity at a second distance, smaller or equal than the first distance, from the geometrical plane comprising the second surface of the housing, and wherein at least a portion of the second surface of the housing comprises a skin adhesive.

Other means for maintaining said electrical contacting unit in a resiliently deformed state when in contact with said skin may comprise a body part fitting device (e.g. having a clamping system). An example of such a body part fitting device is a headset with a clamping system.

In an embodiment, said second distance between the first surface of the electrode and the geometrical plane comprising the second surface of the housing is larger than 0 mm, preferably larger than 0.1 mm, more preferably larger than 0.5 mm. Preferably, it is smaller than 2 mm, more preferably smaller than 1.5 mm. Any combination of a lower limit indicated above with a higher limit indicated above is an embodiment of the present disclosure. For instance, this second distance D′ can be defined as follow: 0<D′<2 mm.

In an embodiment, said electrical contacting unit may be secured within said cavity.

In an embodiment, the resiliency of said electrical contacting unit may be at least partly due to it being at least partly made of a resilient material. The use of one or more springs in the structure of the electrical contacting unit is an alternative.

For instance, the resiliency of said electrical contacting unit may be due to the resilient material from which it is made.

In some embodiments of the disclosure, the resilient material comprises a rubber or an elastomer. A resilient material can undergo numerous elastic deformations under stress and still return to its original size without permanent deformation. Also materials with a foam basis which are resilient can be used.

In some embodiments, the resilient material may have a Young's modulus of from 30 KPa to 50 MPa, preferably from 35 KPa to 30 MPa.

In yet other embodiments of the disclosure, the resilient material is polydimethylsiloxane.

In an embodiment, said electrical contacting unit may be within the cavity in such a way that the first surface of said electrode is substantially parallel to said second surface of said housing.

As used herein and unless provided otherwise, the term “skin adhesive” relates to an adhesive permitting adhesion to the skin.

Preferably, the skin adhesive is selected so that it does not cause a reaction with the skin, e.g. Cleartrace™ Adult ECG Electrode Stickers can be used.

The first surface S may bear means for lessening movements between the skin and said first surface. Examples of such means are a castellated structure and/or skin piercing microprojections.

In some embodiments of a sensor in accordance with the disclosure, the at least one electrode may have a castellated cross-section.

As used herein and unless provided otherwise, by castellated cross-section it is meant a cross-section defined by alternate solid parts (merlons) and open spaces (crenels). The solid parts preferably each have a first surface S (at the top of said solid part), the ensemble of said first surfaces being in a same geometrical plane. The top surfaces may bear further means, e.g. skin piercing microprojections, for lessening movements between the skin and said first surface. The open spaces (or crenels) may have any shapes but are typically of rectangular or trapezoidal cross-section. They make space for accommodating hairs, thereby allowing the first surfaces to get more easily into close contact with the skin.

The castellated cross-section advantageously offers a better contact between the electrodes and the skin when the sensor is used with hairy skin. In addition, the castellated shape hinders relative lateral movements between the skin and the electrode. When the electrode attempts to move relative to the skin, the corner of the merlons may dig into the skin thereby creating friction between the electrode and the skin, thereby reducing the motion artifact.

As mentioned above, in some embodiments of the disclosure, the first surface may bear means for lessening movements between the skin and the first surface. Preferably, the means for lessening movements comprise at least one and preferably a plurality of skin piercing microprojections. Preferably, these skin piercing microprojections are suitable for only penetrating the stratum corneum layer of the skin. Preferably, the skin piercing microprojections have a height of from 0.1 to 40 μm, preferably from 0.1 to 20 μm, more preferably from 0.1 to 10 μm and most preferably from 0.1 to 5 μm. The microprojections may for instance be needles

An advantage of these embodiments is that these microprojections have such dimensions that they are penetrating only the stratum corneum, both minimizing the contact impedance and the possibility of relative lateral movement between the electrode and the skin. In addition, the stratum corneum does not contain any blood vessel or nerve; hence no bleeding, infection or pain sensation will be caused by the microprojections.

In embodiments of the disclosure, the resiliency of the electrical contacting unit may be at least partly due to it comprising a resilient substrate. For instance, the electrical contacting unit may comprise a resilient substrate wherein the at least one electrode is secured on the resilient substrate in such a way that the first surface is facing away from the resilient substrate, and wherein the resilient substrate is secured to said housing. In this embodiment, the resiliency of the electrical contacting unit is partly or entirely provided by the resiliency of the substrate. This is advantageous as it permits to use non-resilient materials for the electrodes.

In an embodiment, the first distance D can be larger than 0 mm, preferably larger than 0.1 mm, more preferably larger than 0.5 mm. Preferably, it is smaller than 2 mm, more preferably smaller than 1.5 mm. Any combination of a lower limit indicated above with a higher limit indicated above is an embodiment of the present disclosure. For instance, this first distance D can be defined as follow: 0<D<2 mm.

The thickness of the substrate is preferably at least equal and most preferably larger than the second distance D′. In an embodiment, if the electrical contact unit is pressed against the skin, it is the substrate that can resiliently deform over a first distance D in the direction of said pressure P. For instance, the total thickness T of the substrate can be defined as follow: D+0.5 mm<T<D+1.5 mm.

Such a thickness is advantageous as it permits on one hand to allow the resilient deformation of the substrate over a distance of at least D′ and on another hand, it permits to keep the substrate flexible enough to mimic the skin flexibility.

In some embodiments in accordance with the disclosure, the sensor may further comprise electrical connection means for connecting the at least one electrode to signal output means such as e.g. a signal output line. In an embodiment, the electrical connection means may be interconnects embedded in the resilient substrate.

In yet other embodiments in accordance with the disclosure, the resilient material may be conductive, thereby providing the electrical connection means. An example of conductive resilient material is a conductive rubber.

In other embodiments of the disclosure, electrically conductive elastomers can be used, for instance by incorporating highly structured carbon blacks, conductive plasticizers and/or other electrically conductive additives to the elastomers. U.S. Pat. No. 4,317,265 discloses such an elastomer.

In an embodiment in accordance with the disclosure the at least one electrode may be at least one dry electrode. In the prior art, gel material has been used at the interface between a conventional electrode and the skin in order to promote a smooth electrical contact. Such gels, however, causes an uncomfortable sensation and in some cases may cause skin irritation. The present disclosure assures a contact between the electrodes and the skin which is good enough to make optional the use of a gel. This kind of electrode does not require a conductive paste, for instance a wet gel at the interface with the skin. A dry electrode has the advantages of not needing to prepare the skin or using conductive paste, reducing the sensitivity to motion artifacts and enabling an enhanced signal-to-noise ratio. Dry electrodes can be used as contact type electrodes, when an electrode-skin contact is established, or as non-contact type electrodes, wherein a capacitive coupling with the skin is created.

An advantage of embodiments of the present disclosure is that dry contact electrodes are inherently less prone to motion artifact than dry non-contact type electrodes which operate with capacitive coupling.

In another embodiment in accordance with the disclosure the at least one electrode may be coated with an impedance reducing coating. Preferably the impedance reducing coating is a biocompatible material such as PEDOT or IrOx.

When using dry contact electrodes with no wet gel on the electrodes, the contact impedance can be reduced by the use of such a coating, thereby also reducing the motion artifact.

In some embodiments in accordance with the disclosure the biopotential measurement performed by the sensor may be an electrocardiographic measurement or an electroencephalographic measurement.

In embodiments in accordance with the disclosure, the at least one electrode may be a plurality of electrodes. Preferably, the pluralities of electrodes may be electrically connected in parallel.

The use of a plurality of electrodes is especially advantageous when they are present on a flexible substrate.

An advantage of having a plurality of electrodes is the high degree of flexibility created by the plurality of electrodes when present on a flexible substrate. The flexibility obtained by the assembly electrodes-flexible substrate, can be similar to that of human skin, thereby improving the electrode-skin contact and decreasing noise by motion artifacts.

In embodiments, the electrodes themselves can be made of a rigid material. In such a case, there is a mismatch with the skin which is soft and elastic. Placing a single electrode on a flexible substrate is not improving much this situation since the electrode itself remains rigid. However, using a plurality of electrodes on a flexible substrate permits to have a sensor that is mimicking the flexibility of the skin and which permit a better electrode-skin contact.

Another advantage of using a plurality of electrodes which are electrically connected in parallel with one another is the lower impedance one obtains compared to using one big rigid planar electrode.

In an embodiment of the disclosure the plurality of electrodes are from 2 to 16 electrodes.

In another embodiment of the disclosure the ensemble of the at least one electrode is confined in maximum 3 cm² (e.g. in maximum 1 cm²). This is advantageous since it permits the different electrodes of a particular sensor to all measure the same location of the body.

In other words, instead of using one big planar electrode, a plurality of electrodes which function in parallel will result in lower contact impedance. Preferably, from 2 to 16 electrodes can be used. In addition, an electrical contacting unit comprising a multiplicity of electrodes 3 attached to a flexible substrate 5 enables the mimicking of the skin's flexibility. The electrodes 3 are preferably placed close to each other as they should pick up the same biopotential signal. Also, when the electrodes are placed too far apart important impedance variations could be induced between the electrodes due to variations in local skin stretching.

In a second aspect, the present disclosure relates to a device comprising one or more sensors according to the first aspect of the present disclosure.

In a third aspect of the present disclosure, one or more electrical contacting unit as described in any embodiment of the first aspect of the present disclosure can be secured in a headset. For instance, the headset may comprise a left band further comprising a left ear notch configured to position about a subject's left ear and facilitate placement of the electrode headset on the subject's head; and the right band further comprises a right ear notch configured to position about a subject's left ear and facilitate placement of the electrode headset on the subject's head.

The one or more electrical contacting units mounted in the headset can be properly positioned relative to the subject's head and in accordance with a desired placement scheme.

The headset is preferably adapted to create a pressure on the electrical contacting units, thereby ensuring that the electrodes remain in a substantially stable position throughout use. This can for instance be achieved by providing the headset with a clamping system.

The combination of the electrical contacting units having a resilient character and the headset providing pressure, provide a good contact at the electrode-scalp interface which can allow noise to settle relatively quickly, and a clean signal can be achieved relatively quickly as compared to prior art headsets.

In a fourth aspect, the present disclosure relates to the use of a sensor according to the first aspect or to a headset according to the second aspect for measuring a biopotential such as a EEG or a ECG.

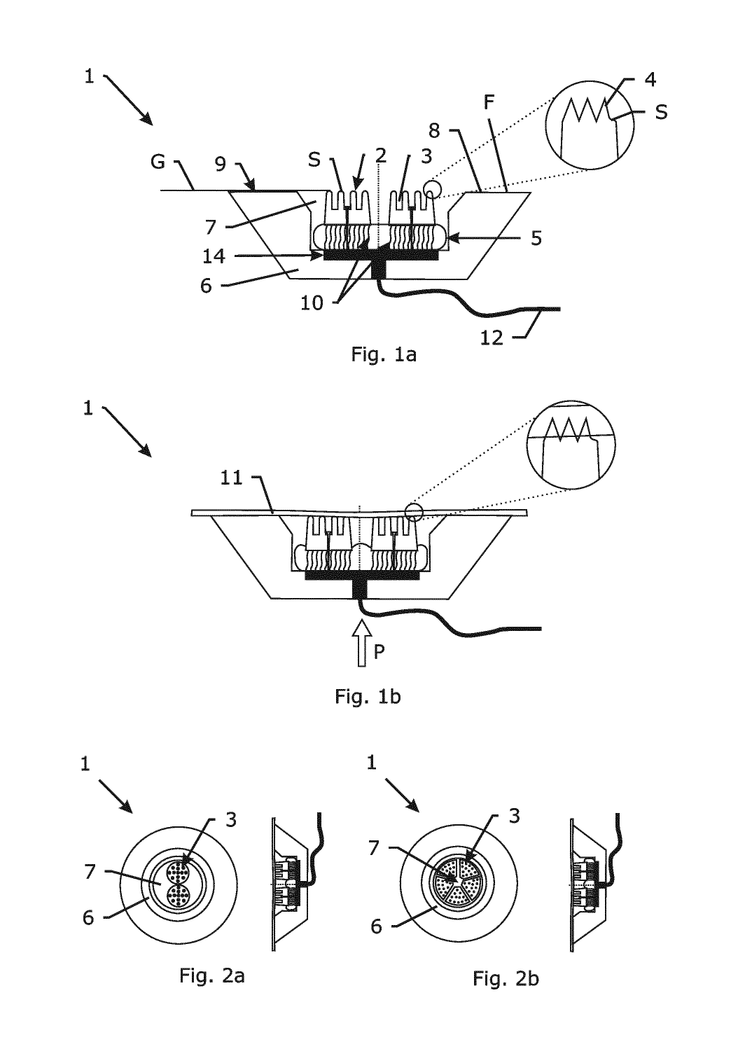

FIGS. 1a and 1b show schematic representations of the cross-section of a sensor 1 for biopotential measurement, according to an embodiment of the disclosure. The sensor 1 comprises an electrical contacting unit 2 and a housing 6. FIG. 1a schematically shows the global layout and construction of the sensor and FIG. 1b schematically shows the functioning of the sensor.

In the embodiment of FIG. 1a, an electrical contacting unit 2 is shown It comprises a resilient substrate 5, two electrodes 3, and electrical connection means (interconnects 10). Two needles 15 for securing each electrode to the resilient substrate 5 and for securing the substrate 5 to the housing 6 are also shown.

Item 14 is a conducting plate embedded in the substrate.

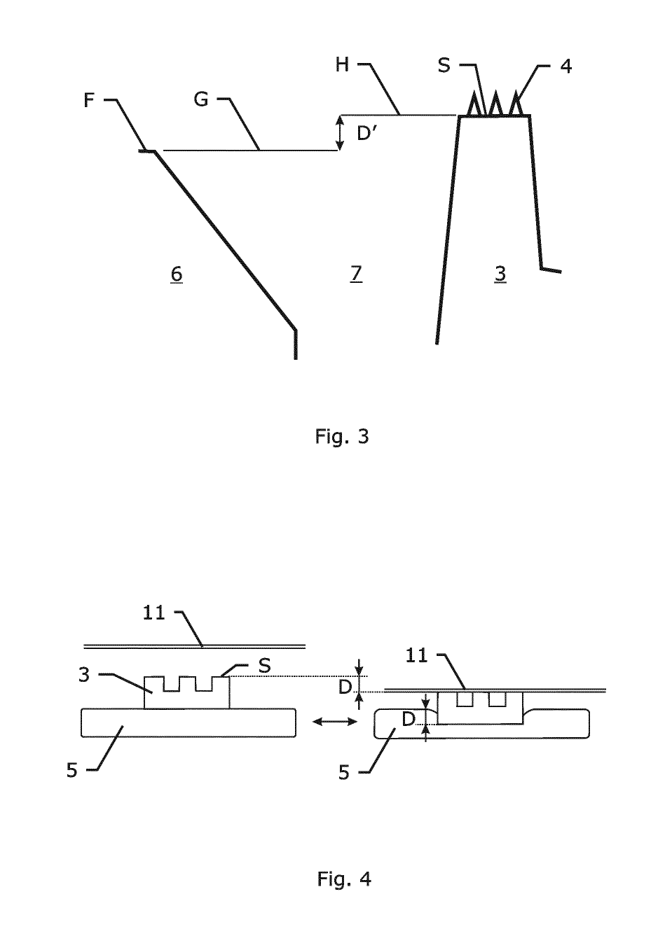

Each electrode 3 comprises a surface S for contacting the skin. The housing 6 of the sensor 1 shown in FIG. 1a comprises a second surface F and a cavity 7 within this second surface F. At least a portion 8 of the second surface F comprises a skin adhesive 9. The electrical contacting unit 2 is secured within the cavity 7 of the housing 6. The securing of the electrical contacting unit 2 is such that the surface of the electrode S is substantially parallel to the surface F of the housing 6. Thus, the surface of each electrode S is at an equal distance from the surface F of the housing. Moreover the surface S of the electrode is positioned at a distance D′ outside the cavity 7 of the sensor compared to the geometrical plane G comprising the second surface F. D′ is shown in more details in FIG. 3.

FIG. 1b schematically shows the sensor according to the embodiment of FIG. 1a in use. Due to the positioning of the electrodes at a distance D′ above the surface F, the resilient substrate 5 deforms when the surface F is contacted with the skin 11. Due to the presence of the skin adhesive 9, the sensor is maintained in contact with the skin and the electrodes are pressed onto the skin.

Using the adhesive zone surrounding the electrodes, the created pressure P press the electrodes 3 firmly against the skin 11, due to the presence of the resilient electrical contacting unit 2. This does not cause discomfort to the patient.

The contact between the electrode 3 and the skin 8 can be ameliorated by means for lessening the movements between the skin 11 and surface (S) of the electrode 3. At least one or preferably a plurality of skin piercing microprojections 4 can be used to enhance the electrode-skin contact. The enlarged portion of FIG. 1b shown on the right side of the figure shows that the microprojections enter the skin. The electrode 3 is secured on the resilient substrate 5 which is secured to the housing of the sensor 6. This securing can be done in such a way that the projections 4 are pointing away from the resilient substrate 5. The securing of the electrode 3 on the resilient substrate can be enabled by for instance a needle and/or by providing an intermediate bounding layer e.g. polyimide. In some embodiments of the present disclosure, the electrode and resilient substrate can be assembled from one single piece and thus not needing any means for securing the electrode on the resilient substrate. The microprojections 4 can be small micro needles that preferably only penetrate the stratum corneum. Because only the stratum corneum is penetrated, which is the outermost layer of the epidermis, composed of large, flat, polyhedral, plate-like envelopes filled with keratin and largely made up of dead cells that have migrated up from the stratum granulosum, no bleeding, infection or pain sensation will be caused by the micro needles. In addition, by using the micro needles, the contact impedance of the electrodes is minimized. In addition the microprojections maximize the skin 11 attachment of the electrode 3 providing a reduction of the motion artifact.

The castellated cross-section of the sensor 1 additionally provides a better electrode-skin contact. The indentations, which can be similar to battlements, enables the hair on the skin 11 to move through them providing a lower contact impedance and additionally decrease the sheer movement against the skin 11 and therefore improving the electrode-skin contact.

As described above preferably a rubber or an elastomer is suitable to use as a resilient material for the electrical contacting unit 2. To enable the transport of the biopotential signal measured by the electrode 3, electrical connection means 10 for connecting the electrode 3 to a signal output line 11 can be provided in the resilient substrate 5. Preferably interconnects 10 embedded in the resilient substrate 5 can be used for transporting the signal. If the substrate comprises a conductive material, interconnects are not required since the conductive nature of the material enables the transportation of the biopotential signal.

FIG. 2a and FIG. 2b are a schematic representation of different configurations of a sensor according to an embodiment of the disclosure. The left figures in FIG. 2a and FIG. 2b show a top-view of a sensor comprising an ensemble of the at least one electrode 3 (two electrodes in the case of FIGS. 2a and 5 electrodes in the case of FIG. 2b). The right figures in FIG. 2a and FIG. 2b show a cross-section of the sensor. The ensemble can have various configurations comprising different geometries and different sizes, for instance circular or quadrangle as illustrated in FIGS. 2a and 2b, respectively. Moreover, the ensemble of the one or more electrodes preferably is confined in maximum 1 cm².

FIG. 3 shows an enlarged portion of FIG. 1a. Visible in the figure are a substrate 6 with a second surface F and a cavity 7 within said second surface F. Also visible is an electrode 3. The electrode 3 has a surface S and microprojections 4. This figure defines the distance D′ as being the distance between the first surface S and the second surface F. In other words, D′ is the distance between the geometric plane H comprising the first surface S and the geometric plane G comprising the second surface F.

FIG. 4 shows on its left side an electrical contact unit before being pressed against a skin 11. It comprises a resilient substrate and an electrode 3 having a surface S. On the right side of the figure, the electrical contacting unit is shown when in contact with the skin 11. It shows a deformation of the resilient substrate by a distance D and therefore a resilient deformation of the electrical contact unit by this same distance D.

It is to be understood that the disclosure is not limited to the particular features of the means and/or the process steps of the methods described as such means and methods may vary. It is also to be understood that the terminology used herein is for purposes of describing particular embodiments only, and is not intended to be limiting. It must be noted that, as used in the specification and the appended claims, the singular forms “a” “an” and “the” include singular and/or plural referents unless the context clearly dictates otherwise. It is also to be understood that plural forms include singular and/or plural referents unless the context clearly dictates otherwise. It is moreover to be understood that, in case parameter ranges are given which are delimited by numeric values, the ranges are deemed to include these limitation values.

The particular combinations of elements and features in the above detailed embodiments are exemplary only. As those skilled in the art will recognize, variations, modifications, and other implementations of what is described herein can occur to those of ordinary skill in the art without departing from the spirit and the scope of the disclosure as claimed. Accordingly, the foregoing description is by way of example only and is not intended as limiting. The disclosure's scope is defined in the following claims and the equivalents thereto. Furthermore, reference signs used in the description and claims do not limit the scope of the disclosure as claimed.

1. A sensor configured for biopotential measurement, comprising:
an electrical contacting unit for establishing an electrical contact with skin, wherein the electrical contacting unit (i) can resiliently deform over a first distance in the direction of pressure in response to being pressed against the skin and (ii) comprises one or more electrodes, wherein each of the one or more electrodes comprises a first surface for contacting the skin, and wherein the skin is mammalian skin; and
a housing comprising a second surface and a cavity within the second surface, wherein
the electrical contacting unit is positioned within the cavity such that the first surface of each of the one or more electrodes is outside of the cavity at a second distance from a geometrical plane comprising the second surface of the housing, wherein the second distance is less than or equal to the first distance, and
a skin adhesive is present on at least a portion of the second surface of the housing.

2. The sensor according to claim 1, wherein the electrical contact unit is at least partially made of a resilient material, wherein a resiliency of the electrical contacting unit is at least partially due to the resilient material.

3. The sensor according to claim 1, wherein at least one of the one or more electrodes comprises a castellated cross-section.

4. The sensor according to claim 1, wherein the first surface includes means for reducing movement between the skin and the first surface.

5. The sensor according to claim 4, wherein the means for reducing movement include at least one skin piercing microprojection.

6. The sensor according to claim 5, wherein the at least one skin-piercing microprojection is configured to penetrate only a stratum corneum layer of the skin.

7. The sensor according to claim 1, wherein the electrical contacting unit further comprises a resilient substrate, wherein
(i) each of the one or more electrodes is secured to the resilient substrate such that the first surface faces away from the resilient substrate,
(ii) the resilient substrate is secured to the housing, and
(iii) a resiliency of the electrical contacting unit is at least due in part to the resilient substrate.

8. The sensor according to claim 2, wherein the resilient material has a Young's modulus between about 30 KPa and about 50 MPa.

9. The sensor according to claim 1, wherein at least one of the one or more electrodes is a dry electrode.

10. The sensor according to claim 1, wherein the biopotential measurement is one of an electrocardiographic measurement or an electroencephalographic measurement.

11. The sensor according to claim 1, wherein the one or more electrodes comprise a plurality of electrodes.

12. The sensor according to claim 11, wherein the plurality of electrodes are electrically connected in parallel.

13. The sensor according to claim 1, wherein the one or more electrodes are confined to an area that is less than or equal to 3 cm².

14. (canceled)

15. A sensor configured for biopotential measurement, comprising:
an electrical contacting unit for establishing an electrical contact with skin, wherein the electrical contacting unit (i) can resiliently deform over a first distance in the direction of pressure in response to being pressed against the skin and (ii) comprises at least one electrode, wherein the at least one electrode comprises a first surface for contacting the skin, and wherein the skin is mammalian skin;
a housing comprising a second surface and a cavity within the second surface, and
means for maintaining the electrical contacting unit in a resiliently deformed state when in contact with the skin, wherein the electrical contacting unit is positioned within the cavity such that the first surface of the at least one electrode is outside of the cavity at a second distance from a geometrical plane comprising the second surface of the housing, wherein the second distance is less than or equal to the first distance.
